(19) United States
(12) Patent Application Publication
(10) Pub. No.: US 2019/0285649 A1
(43) Pub. Date: Sep. 19, 2019

(54) Biomarker Panel to Identify Patients at Risk for Peri-Implant Osteolysis

(71) Applicant: Rush University Medical Center, Chicago, IL (US)

(72) Inventors: Ryan D. Ross, Chicago, IL (US); Dale Richman Sumner, Chicago, IL (US); Joshua J. Jacobs, Chicago, IL (US)

(73) Assignee: Rush University Medical Center, Chicago, IL (US)

(21) Appl. No.: 16/357,578

(22) Filed: Mar. 19, 2019

Related U.S. Application Data
(60) Provisional application No. 62/644,716, filed on Mar. 19, 2018.

(51) Int. Cl. G01N 33/68

(52) U.S. Cl. CPC G01N 33/6893; G01N 2800/10

(57) ABSTRACT

Methods and kits for measuring a panel of biomarkers in a subject suspected of being at risk for peri-implant osteolysis are provided. The method includes obtaining a biological sample from the subject; and measuring a level of at least two biomarkers in a biomarker panel in the sample, wherein the biomarker panel comprises α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX).

### RELATED APPLICATIONS

This application claims the benefit of U.S. Provisional Application No. 62/644,716, filed Mar. 19, 2018, which is incorporated by reference herein in its entirety.

### FEDERALLY SPONSORED RESEARCH OR DEVELOPMENT

This invention was made with government support under federal grant number R01AR066562 awarded by National Institute of Arthritis and Musculoskeletal and Skin Diseases (NIAMS). The government has certain rights in the invention.

### TECHNICAL FIELD

The present disclosure relates to methods and kits for identifying subjects at risk for peri-implant osteolysis, and in particular to methods and kits for identifying subjects at risk for peri-implant osteolysis and for determining optimal treatment plans for subjects at risk for peri-implant osteolysis.

### BACKGROUND

The estimated number of patients in need of primary total joint replacement (TJR) surgery in the United States is projected to grow to over 4 million annually by the year 2030¹. Revision surgeries are expected to grow at a similar rate, with more than 350,000 revision surgeries anticipated in the U.S. in the year 2030¹. Aseptic loosening is one of the primary mechanisms contributing to failure of primary TJR². Peri-implant osteolysis, induced by wear or corrosion products, is a key factor contributing to aseptic loosening³⁻⁵. Peri-implant osteolysis can progress asymptomatically, resulting in substantial bone loss, until mechanical failure of the bone-implant interface gives rise to symptoms (i.e. patient reported pain). Clinically, plain radiography is typically used for diagnosis, but detection of small osteolytic lesions is better served by cross-sectional imaging⁶. Currently, revision surgery is the only treatment for aseptic loosening caused by osteolysis. Revision surgeries are associated with higher failure rates⁷, higher patient mortality rates⁸, and worse pain and functional outcomes⁹ than primary TJR. Substantial bone loss prior to revision surgery is a contributing factor to these poorer outcomes¹⁰.

Diagnosing osteolysis prior to substantial bone loss could provide surgeons with the opportunity to perform revision surgery while the patient still maintains adequate bone mass. Additionally, preclinical studies have shown that biological and pharmacological treatment strategies are able to prevent osteolysis progression if initiated early^{11-13.} Unfortunately, clinical trials to test early intervention strategies are lacking. Bisphosphonates, when given late in disease progression, have been tested clinically without success¹⁴. Interestingly, a registry study found that patients receiving long-term bisphosphonate treatment have a reduced rate of revision surgery¹⁵, implying that early intervention with this class of drugs may be an effective treatment strategy. One reason that few clinical trials have been initiated is the inability to identify patients at risk for osteolysis prior to radiographic diagnosis or patient reported pain.

Systemic biomarkers have shown considerable promise as a means to diagnose or monitor the response to treatment in musculoskeletal diseases such as osteoporosis¹⁶. The use of noninvasive or minimally invasive biomarkers to monitor patients post-surgery would provide real-time information on peri-implant inflammation or bone resorption, which in turn could be used to identify patients at risk for osteolysis. To date, biomarker studies in patients have been inconsistent and at times contradictory¹⁷. The major limitation of current biomarker research is that most studies only assess marker levels at the end-stage of osteolysis. Therefore, there is little evidence that biomarkers can be used to predict osteolysis prior to implant failure. A single study has measured marker levels longitudinally and although the authors used early implant migration as a surrogate for eventual implant loosening, two of the biomarkers evaluated demonstrated early differences between stable and migrating implants¹⁸.

### BRIEF SUMMARY

Methods and kits for measuring a panel of biomarkers in a subject suspected of being at risk for peri-implant osteolysis are provided. The method includes obtaining a biological sample from the subject; and measuring a level of at least two biomarkers in a biomarker panel in the sample, wherein the biomarker panel comprises α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX).

Methods of treating a subject at risk for or having peri-implant osteolysis are also provided. The methods include obtaining or having obtained a biological sample from the subject and performing or having performed a measurement of a level of at least two biomarkers in a biomarker panel in the sample, wherein the biomarker panel comprises α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX). The methods also include administering to the subject at risk for or having peri-implant osteolysis a therapeutically effective amount of a therapeutic agent selected from the group consisting of anti-catabolic agents to reduce further bone loss or anabolic agents to increase bone formation around the implant.

### BRIEF DESCRIPTION OF THE DRAWINGS

FIGS. 1a-1g illustrate urinary concentrations of 1a) α-CTX, 1b) IL-6, 1c) DPD, 1d) OPG, 1e) NTX, 1f) β-CTX, and 1g) IL-8 normalized to urinary creatinine and the creatinine normalized pre-operative concentration in the non-osteolysis group (white bars) and osteolysis (shaded bars) groups. The data are presented as the median (middle of box), 25^{th} to 75^{th} percentile (hinges of box), and the minimum and maximum values (whiskers). Between-group differences (p<0.05, Mann-Whitney U test) for specific time-points are indicated by a horizontal bar.

FIGS. 2a-2g illustrate individual biomarker plots showing the change in creatinine and pre-operatively normalized 2a) α-CTX, 2b) IL-6, 2c) DPD, 2d) OPG, 2e) NTX, 2f) β-CTX, and 2g) IL-8 normalized to urinary creatinine and the creatinine normalized pre-surgery concentration in the non-osteolysis and osteolysis groups.

FIG. 3 shows Table 3 listing individual biomarker concentrations as a function of time prior to diagnosis.

### DETAILED DESCRIPTION

The present disclosure will utilize at least one biomarker measured in a biological sample obtained from a subject to identify subjects at risk for peri-implant osteolysis. In some embodiments, the at least one biomarker may be selected from a panel of biomarkers. In some embodiments, one or more biomarkers from a panel of biomarkers are used to identify subjects having at risk for peri-implant osteolysis.

The term “biomarker” as used herein, refers to any biological compound that can be measured as an indicator of the physiological status of a biological system. A biomarker may comprise an amino acid sequence, a nucleic acid sequence and fragments thereof. Exemplary biomarkers include, but are not limited to cytokines, chemokines, growth and angiogenic factors, inflammation, bone resorption products, apoptosis related proteins, proteases, adhesion molecules, cell signaling molecules and hormones.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “detecting” or “detection” may be used and is understood to cover all measuring or measurement as described herein.

“Osteolysis” refers to an active resorption of bone matrix by osteoclasts. Osteolysis often occurs in the proximity of a prosthesis, likely as a consequence of an immunologic response to wear or corrosion products shed from the prosthesis. Ongoing osteolysis can elicit changes in the structural integrity of the bone and/or the bone-prosthesis interface.

The terms “sample” or “biological sample” as used herein, refers to a sample of biological fluid, tissue, or cells, in a healthy and/or pathological state obtained from a subject. Such samples include, but are not limited to, blood, bronchial lavage fluid, sputum, saliva, urine, amniotic fluid, lymph fluid, tissue or fine needle biopsy samples, peritoneal fluid, cerebrospinal fluid, and includes supernatant from cell lysates, lysed cells, cellular extracts, and nuclear extracts. In some embodiments, the whole blood sample is further processed into serum or plasma samples. In some embodiments, the sample includes urine.

The term “subject” or “patient” as used herein, refers to a mammal, preferably a human.

### Biomarkers

Biomarkers that may be used include but are not limited to cytokines, chemokines, growth and angiogenic factors, inflammation, bone resorption products, apoptosis related proteins, proteases, adhesion molecules, cell signaling molecules and hormones. In some embodiments, the biomarkers may be proteins that are circulating in the subject that may be detected from a fluid sample obtained from the subject. In some embodiments, the fluid sample may be urine. In some embodiments, one or more biomarkers from a panel of biomarkers may be used. In some embodiments, the one or more biomarker may be measured before or after a treatment, such as a total joint replacement (TJR) or a pharmaceutical treatment designed to inhibit bone resorption or promote bone formation.

In some embodiments, one or more biomarkers may be measured in a biomarker panel. The biomarker panel may include a plurality of biomarkers. In some embodiments, the biomarker panel may include ten or fewer biomarkers. In yet other embodiments, the biomarker panel may include 1, 2, 3, 4, 5, 6 or 7 biomarkers. In some embodiments, the biomarker panel may be optimized from a candidate pool of biomarkers. In some embodiments, the biomarker panel may include two biomarkers. By way of non-limiting example, the biomarker or biomarker panel may be configured for determining whether a subject is at risk for peri-implant osteolysis.

In some embodiments, the biomarker panel may include biomarkers from several biological pathways. By way of non-limiting example, the biomarkers may be selected from free deoxypyridinoline (DPD), cross-linked N-telopeptides (NTX), interleukin-6 (IL-6), interleukin-8 (IL-8), osteoprotegerin (OPG), α-crosslaps (α-CTX), β-crosslaps (β-CTX), TRAP 5b, cathepsin K, RANKL and osteocacin. In some embodiments, the biomarker panel may include DPD, NTX, IL-6, IL-8, OPG, α-CTX, and β-CTX. In yet other embodiments, the panel may include 6, 5, 4, 3 or 2 biomarkers selected from DPD, NTX, IL-6, IL-8, OPG, α-CTX, and β-CTX. In other embodiments, the biomarker panel may include α-CTX and IL-6. In some embodiments, a single biomarker selected from may be used. In certain embodiments, DPD may be used as a single biomarker.

### Biomarker Measurement

Measurement of a biomarker generally relates to a quantitative measurement of an expression product, which is typically a protein or polypeptide. In some embodiments, the measurement of a biomarker may relate to a quantitative or qualitative measurement of nucleic acids, such as DNA or RNA. The measurement of the biomarker of the subject detects expression levels of one or more biomarkers in subjects.

Expression of the biomarkers may be measured using any method known to one skilled in the art. Methods for measuring protein expression include, but are not limited to a multiplex immunoassay using magnetic beads to simultaneously measure multiple analytes in a single experiment, Enzyme-linked immunosorbent assay (ELISA), Western blot, immunoprecipitation, immunohistochemistry, Radio Immuno Assay (RIA), radioreceptor assay, proteomics methods, mass-spectrometry based detection (SRM or MRM) or quantitative immunostaining methods. Methods for measuring nucleic acid expression or levels may be any techniques known to one skilled in the art. Expression levels from the one or more biomarkers are measured in the subject and compared to the levels of the one or more biomarkers obtained from a cohort of subjects described below. Other measurement systems and techniques may also be used.

In some embodiments, a kit may be provided with reagents to measure at least one biomarker. In some embodiments, the kit may be provided with reagents to measure at least two biomarkers in a panel of biomarkers. In some embodiments, the kit may be a multiplex immunoassay. The panel of biomarkers to be measured with the kit may include reagents to measure two or more biomarkers selected from DPD, NTX, IL-6, IL-8, OPG, α-CTX, and β-CTX. In some embodiments, the kit may include reagents for ELISA measurement of the two or more biomarkers. In some embodiments, the kit may include reagents for ELISA measurement of the two or more biomarkers and reagents for measurement of creatinine levels.

### Analysis of Biomarker Measurements

In some embodiments, methods for determining whether a subject is at risk for peri-implant osteolysis may be based upon the biomarker measurements from the subject. In some aspects, the biomarker measurements may be normalized to creatinine and/or pre-operative values. In some embodiments, the predictive value of the panel of biomarkers may be assessed using classification techniques, including but not limited to, logistic regression, Naïve Bayes, Decision Tree and Random Forests.

### Treatment Stratification

In some embodiments, the analysis of the biomarker panel may be used to determine a treatment regime for the subject. In some embodiments, the measurement of one or more biomarkers in the panel may be used to determine the subject is at risk for peri-implant osteolysis before conventional radiographic diagnosis. Subjects at risk for peri-implant osteolysis may begin a treatment or clinical monitoring regime prior to substantial bone loss that is detectable by radiographic diagnosis. Examples of potential treatment strategies include but are not limited to anti-catabolic drugs designed to prevent osteolysis development or anabolic drugs designed to promote bone formation around implants. Non-limiting examples of anti-catabolic drugs include bisphosphonates, denosumab (Amgen), Raloxifene, Lasofoxifene (Pfizer), or Calcitonin. Non-limiting examples of anabolic drugs include parathyroid hormone (PTH), teriparatide (Eli Lily), Abaloparatide (Radius), and Romosozumab (Amgen).

### EXAMPLES

### Study Design

The study design was approved by the institutional review board on human research. A signed consent form was obtained from each patient providing permission for storage of biofluids for future research studies.

A total of 26 patients undergoing primary THR by surgeons at Midwest Orthopedics at Rush that were recruited between 1989 and 1997 were used for this study. 24-hour urine samples were used. The surgeon and implant materials varied. All patients received cementless porous coated acetabular components made from titanium alloy and had ultra-high molecular weight polyethylene (UHMWPE) liners. Eight patients received cementless titanium alloy hip stems, while the remaining 18 had cobalt chrome alloy stems (5 cemented and 13 cementless).

Patients had regular radiographic evaluation around the hip stem and acetabulum. Of the patients enrolled, 16 developed radiographic evidence of peri-implant osteolysis either adjacent to the hip stem or acetabular component and were assigned to the osteolysis group. The remaining 10 patients showed no signs of peri-implant osteolysis radiographically over the ˜19 year course of the follow-up period and were assigned to the non-osteolysis group.

Urine samples were synchronized according to the first reported radiographic lesion at either the acetabulum or the stem, which occurred an average of 90 (±29) months post implantation. The sample taken at the time of diagnosis was classified as the diagnosis time-point, pre-operative samples were collected an average of 3 months prior to surgery, and the first post-operative samples were collected an average of 3 months after THR surgery. Samples collected at yearly intervals post-surgery were assigned to time points 6, 5, 4, 3, 2, and 1 year prior to radiographic diagnosis. Samples from non-osteolysis patients were matched according to the time post-implantation.

There were no significant differences between the non-osteolysis and osteolysis groups for sex or age at surgery (Table 1). The sample size was not consistent at each time-point (Table 2). At diagnosis and at the pre-operative time-point, we had the full complement of cases. Most of the missing data at the earlier time-points was due to lack of a 24-hour urine sample. In addition, for two cases in the osteolysis group there were missing data at 6, 5 and/or 4 years

TABLE 1
Patient Demographics

| | Non-osteolysis | | |
|---|---|---|---|
| | (n = 10) | Osteolysis (n = 16) | p-value |
| % Female (# F/total) | 40% (4/10) | 44% (7/16) | 0.854 |
| Mean Age at Surgery in | 60.5 (4.7) | 56.3 (10.1) | 0.082 |
| Years (Standard | | | |
| Deviation) | | | |

TABLE 2
Sample sizes in the stable and osteolysis
groups at each time-point.

| | Pre- | Post- | 6 | 5 | 4 | 3 | 2 | 1 | |
|---|---|---|---|---|---|---|---|---|---|
| | Oper- | Oper- | years | years | years | years | years | years | Diag- |
| | ative | ative | prior | prior | prior | prior | prior | prior | nosis |
| Non- | 10 | 10 | 8 | 9 | 8 | 8 | 8 | 8 | 10 |
| osteolysis | | | | | | | | | |
| Osteolysis | 16 | 15 | 12 | 12 | 14 | 11 | 16 | 13 | 16 |

### Radiographic Review

The date of radiographic diagnosis of osteolysis was obtained from clinical notes taken contemporaneously at the time of evaluation. Radiograph review was performed by a trained observer not involved in the current study according to well-validated techniques. The acetabulum was divided into three zones according to DeLee and Charnley^{19; 20.} The femur was divided into seven zones as described by Gruen²¹. Osteolysis was defined as an area of localized loss of trabecular bone or cortical erosion that was not apparent on the pre-operative or immediate post-operative radiographs^{22; 23}.

### Biomarker Measurement

Urine samples were frozen at −80 ° C. upon collection and subsequently thawed to aliquot for biomarker measurements. Before measurements, samples were manually mixed and centrifuged at 2,000 g for 2 minutes to separate any agglomerates. Urinary concentrations were determined using commercially available ELISA assays. Candidate biomarkers measured included; free deoxypyridinoline (DPD, Quidel, San Diego, Calif.: intra-assay CV 8.4% and inter-assay CV 4.8%), cross-linked N-telopeptides (NTx, Alere, Orlando, Fla., intra-assay CV 19% and inter-assay CV 5%), interleukin-6 (IL-6, Thermo, Waltham, Mass., intra-assay CV<10%, inter-assay CV<10%). interleukin-8 (IL-8, Thermo, Waltham, Mass., intra-assay CV<10%, inter-assay CV<10%). Osteoprotegerin (OPG, Thermo, Waltham, Mass., intra-assay CV<10%, inter-assay CV<12%), α inter-assay CV 9.4%), and β crosslaps (β-CTX, Immunodiagnostics, Tyne & Wear, UK, intra-assay CV 3.9% inter-assay CV6.9%). Creatine concentration was measured in the Rush Clinical Chemistry Laboratory using an Abbot Architect Immunoassay system (Abbott Architect c1600, Chicago, Ill.). All biomarker values were normalized by dividing by time-matched creatinine concentrations to account for kidney function.

### Statistical Analysis

The data were summarized by descriptive statistics: mean and standard deviation for the continuous variable such as age and biomarker concentrations; frequency and percentage for the categorical variable such as gender. Creatinine normalized biomarker concentrations were compared between groups at each time-point using a non-parametric Mann-Whitney U test. We also made between-group comparisons of biomarker values that had been normalized to both the time-matched creatinine and the pre-operative creatinine normalized measures, to account for inter-individual variation. It was determined that pre-operative normalization increased the between-group differences, and therefore all regression modeling was performed using the doubly normalized biomarker values. The ability of individual biomarkers to differentiate patients with osteolysis from patients without osteolysis was evaluated at each time-point using univariate logistic regression modeling. For ease of result interpretation biomarker concentrations were rescaled by each biomarker's interquartile range (IQR, the difference between 75th and 25th percentiles). Odds ratios (ORs) with 95% confidence intervals (CIs) were calculated. The area (AUC) under the Receiver Operating Curve (ROC) was calculated to assess the discrimination power of each biomarker.

The predictive value of the full panel of biomarkers (each weighted at 14.29%) was assessed at each time-point using multiple classification techniques, including logistic regression, Naïve Bayes, Decision Tree and Random Forests. A limited biomarker panel (subset of biomarkers) that was highly correlated with the classification (non-osteolysis or osteolysis) was chosen at the point of radiographic diagnosis. This limited panel was then tested at each time-point using the same classification strategy outlined above. At each time-point, the prediction accuracy, sensitivity, specificity, and AUC for each classification model were calculated. All the analyses were done using SAS 9.4 and WEKA 3.8. A two-sided test was used and a p-value<0.05 was regarded as statistically significant.

### Results

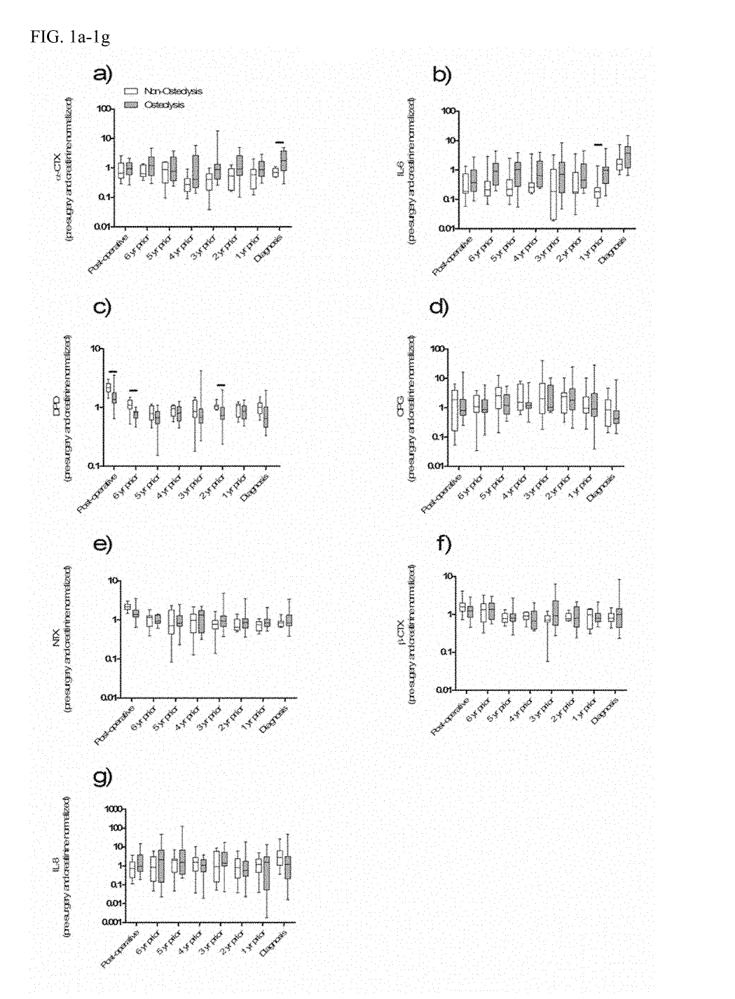

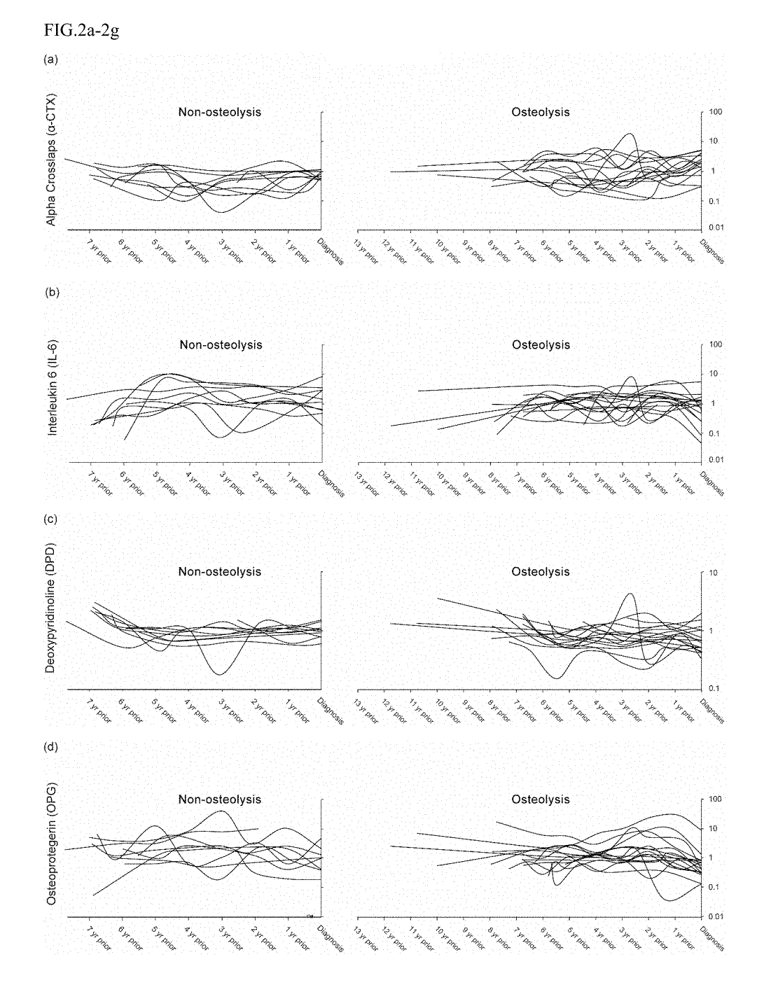

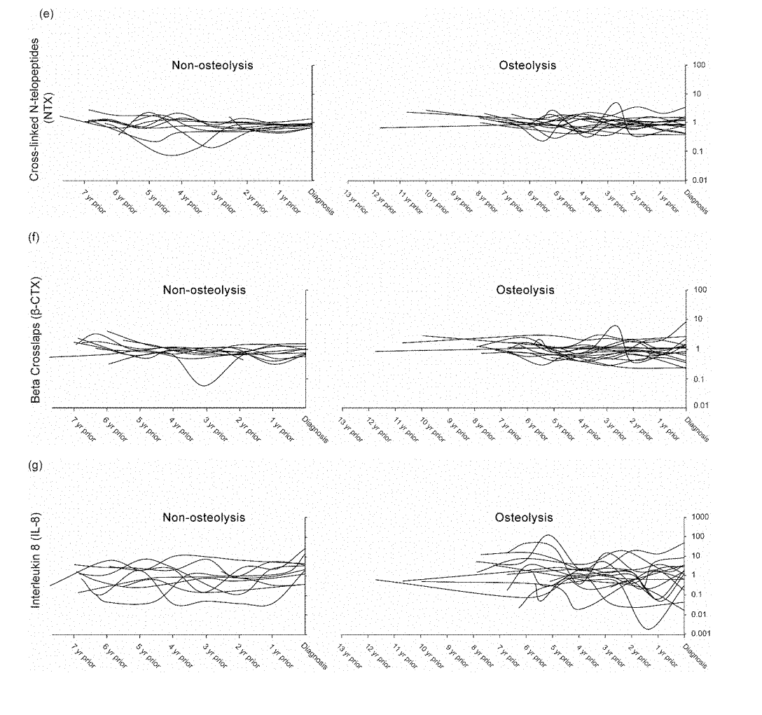

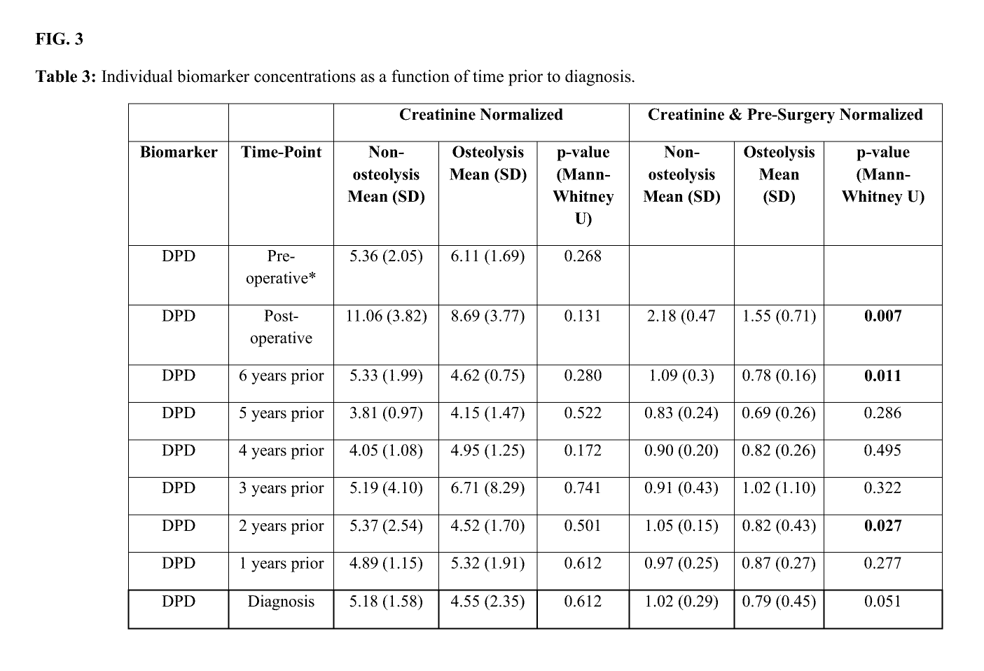

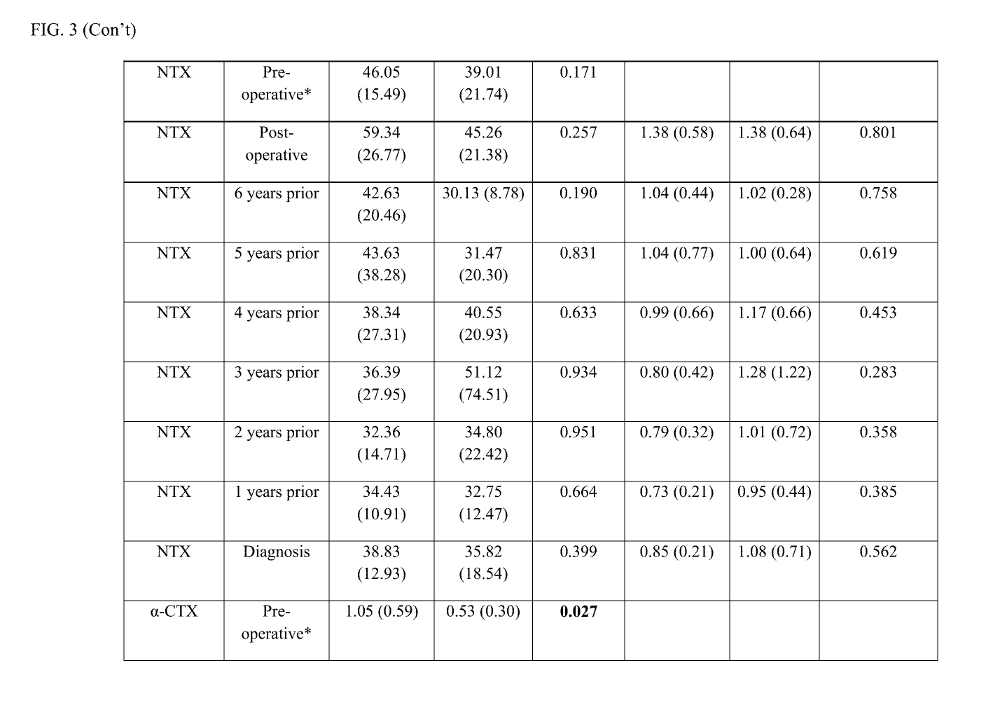

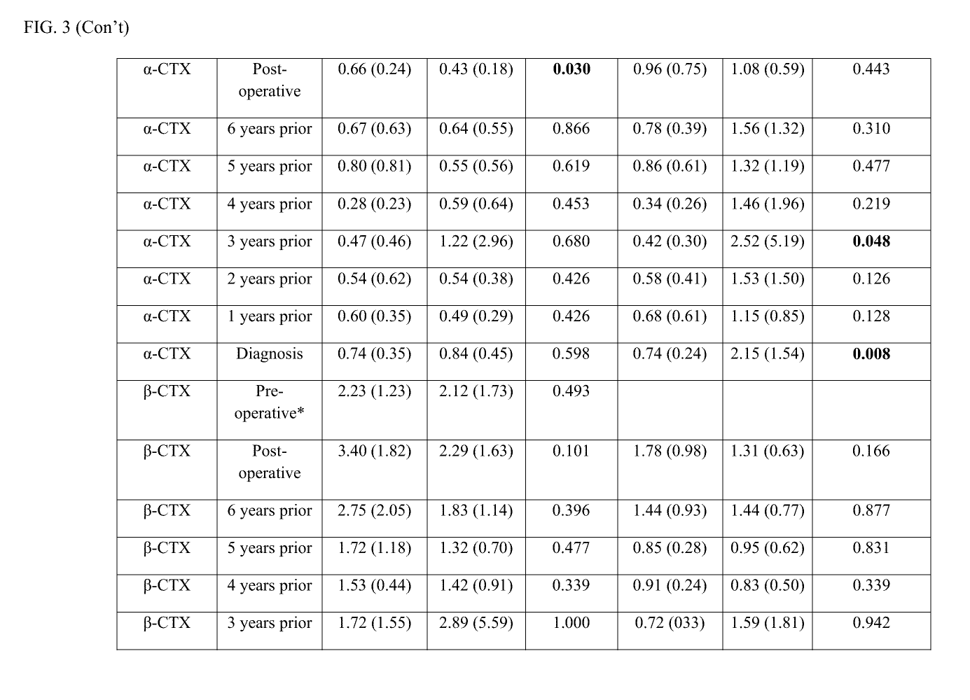

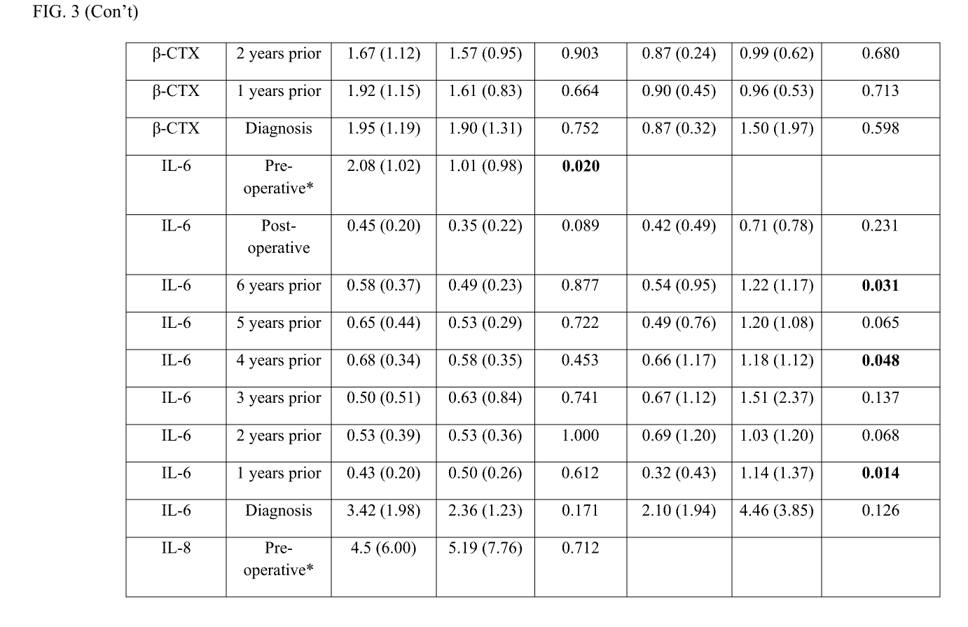

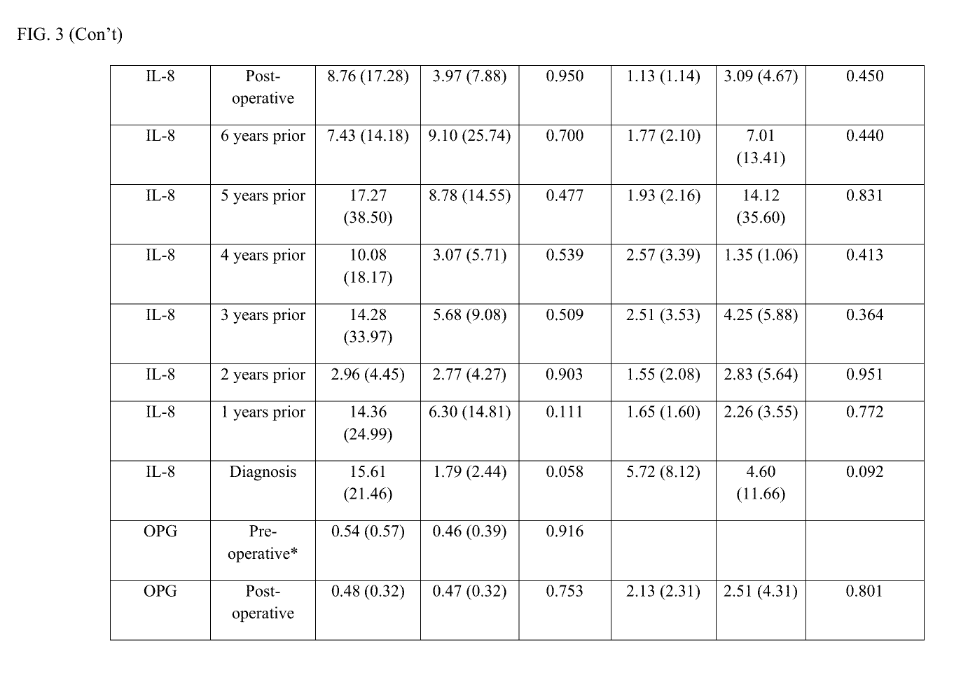

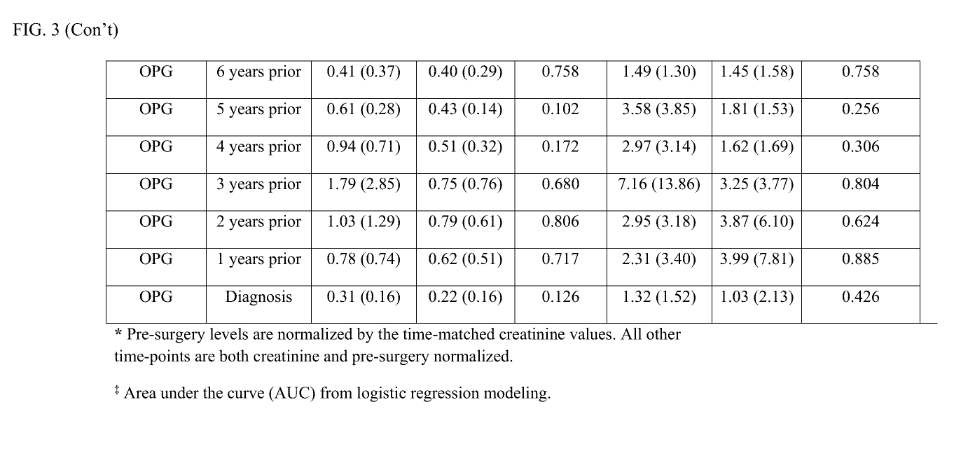

Pre-operative levels of α-CTX (p=0.027) and IL-6 (p=0.020), were significantly depressed in the osteolysis group compared to the non-osteolysis group (Table 3 shown in FIG. 3). Post-operative biomarker concentrations (normalized to the pre-op values) showed occasional between-group differences (FIGS. 1a-1g). Specifically, α-CTX was elevated in the osteolysis group compared to the non-osteolysis controls at 3 years prior to diagnosis (p=0.048) and at diagnosis (p=0.007). IL-6 levels were elevated in the osteolysis group at 6 (p=0.031) and 4 (p=0.048) years prior to diagnosis, and at diagnosis (p=0.014). The DPD concentration was lower in the osteolysis group compared to the non-osteolysis controls post-operatively (p=0.007) and at 6 (p=0.011) and 2 (p=0.027) years prior to diagnosis. OPG, NTX, β-CTX, and IL-8 levels were not different between the osteolysis and the non-osteolysis groups at any post-operative time point. Post-operative biomarker levels for each individual patient are plotted as shown in FIGS. 2a-2g.

The individual biomarker with the highest AUC varied across the time-points investigated (Table 4). At diagnosis and 3 years prior to diagnosis, α-CTX had the highest individual AUC (0.809 and 0.773, respectively). IL-6 had the highest AUC pre-operatively (0.775) and at 1 (0.822), 4 (0.768), and 5 years prior to diagnosis (0.741). The highest individual AUC measured was DPD at 6 years prior to diagnosis (0.844), while the first sampling point after surgery (post-operative) was nearly as high (0.841). AUC results from the univariate logistic regression analysis for all biomarkers at each time-point evaluated are presented in detail in Table 5.

TABLE 4
Univariate logistic regression results showing the biomarker
with the highest AUC for the development of osteolysis at each
of the time-points investigated.

| | | | Odds Ratio | |
|---|---|---|---|---|
| Time-point | Biomarker | Unit (IQR) | (95% CI) | AUC |
| Pre-operative* | IL-6 | 1.821 | 0.161 | 0.775 |
| | | | (0.032, | |
| | | | 0.811) | |
| Post-operative | DPD | 0.862 | 0.234 | 0.841 |
| | | | (0.054, | |
| | | | 1.018) | |
| 6 years prior | DPD | 0.274 | 0.156 | 0.844 |
| | | | (0.026, | |
| | | | 0.933) | |
| 5 years prior | IL-6 | 1.064 | 3.094 | 0.741 |
| | | | (0.699, | |
| | | | 13.70) | |
| 4 years prior | IL-6 | 1.605 | 2.244 | 0.768 |
| | | | (0.462, | |
| | | | 10.90) | |
| 3 years prior | α-CTX | 0.802 | 8.103 | 0.773 |
| | | | (0.600, | |
| | | | 109.4) | |
| 2 years prior | DPD | 0.375 | 0.490 | 0.781 |
| | | | (0.177, | |
| | | | 1.352) | |
| 1 years prior | IL-6 | 0.808 | 6.419 | 0.822 |
| | | | (0.741, | |
| | | | 55.59) | |
| Diagnosis | α-CTX | 1.460 | 19.23 | 0.809 |
| | | | (0.959, | |
| | | | 385.7) | |

*Pre-operative values were normalized to time-matched creatinine values; all other post-operative values were normalized to both creatinine and creatinine-normalized pre-operative values.

TABLE 5
AUC from univariate logistic regression analysis

| Biomarker | Time-Point | AUC^{‡} |
|---|---|---|
| DPD | Pre-operative* | 0.628 |
| DPD | Post-operative | 0.841 |
| DPD | 6 years prior | 0.844 |
| DPD | 5 years prior | 0.639 |
| DPD | 4 years prior | 0.589 |
| DPD | 3 years prior | 0.352 |
| DPD | 2 years prior | 0.781 |
| DPD | 1 years prior | 0.649 |
| DPD | Diagnosis | 0.734 |
| NTX | Pre-operative* | 0.659 |
| NTX | Post-operative | 0.353 |
| NTX | 6 years prior | 0.464 |
| NTX | 5 years prior | 0.458 |
| NTX | 4 years prior | 0.598 |
| NTX | 3 years prior | 0.642 |
| NTX | 2 years prior | 0.613 |
| NTX | 1 years prior | 0.615 |
| NTX | Diagnosis | 0.649 |
| α-CTX | Pre-operative* | 0.759 |
| α-CTX | Post-operative | 0.598 |
| α-CTX | 6 years prior | 0.593 |
| α-CTX | 5 years prior | 0.656 |
| α-CTX | 4 years prior | 0.773 |
| α-CTX | 3 years prior | 0.695 |
| α-CTX | 2 years prior | 0.702 |
| α-CTX | 1 years prior | 0.809 |
| α-CTX | Diagnosis | 0.649 |
| β-CTX | Pre-operative* | 0.581 |
| β-CTX | Post-operative | 0.679 |
| β-CTX | 6 years prior | 0.516 |
| β-CTX | 5 years prior | 0.528 |
| β-CTX | 4 years prior | 0.621 |
| β-CTX | 3 years prior | 0.557 |
| β-CTX | 2 years prior | 0.453 |
| β-CTX | 1 years prior | 0.486 |
| β-CTX | Diagnosis | 0.559 |
| IL-6 | Pre-operative* | 0.775 |
| IL-6 | Post-operative | 0.651 |
| IL-6 | 6 years prior | 0.797 |
| IL-6 | 5 years prior | 0.741 |
| IL-6 | 4 years prior | 0.768 |
| IL-6 | 3 years prior | 0.705 |
| IL-6 | 2 years prior | 0.734 |
| IL-6 | 1 years prior | 0.822 |
| IL-6 | Diagnosis | 0.684 |
| IL-8 | Pre-operative* | 0.450 |
| IL-8 | Post-operative | 0.591 |
| IL-8 | 6 years prior | 0.594 |
| IL-8 | 5 years prior | 0.523 |
| IL-8 | 4 years prior | 0.598 |
| IL-8 | 3 years prior | 0.642 |
| IL-8 | 2 years prior | 0.492 |
| IL-8 | 1 years prior | 0.462 |
| IL-8 | Diagnosis | 0.684 |
| OPG | Pre-operative* | 0.491 |
| OPG | Post-operative | 0.516 |
| OPG | 6 years prior | 0.531 |
| OPG | 5 years prior | 0.648 |
| OPG | 4 years prior | 0.643 |
| OPG | 3 years prior | 0.523 |
| OPG | 2 years prior | 0.492 |
| OPG | 1 years prior | 0.514 |
| OPG | Diagnosis | 0.605 |

*Pre-surgery levels are normalized by the time-matched creatinine values. All other time-points are both creatinine and pre-surgery normalized.
^{‡}Area under the curve (AUC) from logistic regression modeling.

Despite normalizing the post-operative values to creatinine and the pre-operative value, there was considerable variability between patients and within patients over the 6 year follow-up. When the levels were not normalized to the pre-op values, almost none of the post-operative comparisons were significant (Table 3). Therefore, all subsequent logistical modeling was performed using the pre-operative normalized biomarker values.

The use of all seven biomarkers (all normalized to their pre-operative values) in a panel dramatically increased the ability to distinguish the two groups (Table 6). At diagnosis, pre-operatively, and at each time-point post-operatively, the biomarker panel demonstrated high accuracy, sensitivity, and specificity. The random forest classification strategy provided the greatest AUC at each time-point, but the results for each of the other classification strategies are presented in Table 7.

TABLE 6
Accuracy, sensitivity, specificity, and AUC of a full panel of all
seven biomarkers at each time-point investigated using the
Random Forest Classification method.

| Time-point | Accuracy | Sensitivity | Specificity | AUC |
|---|---|---|---|---|
| Pre- | 1.000 | 1.000 | 1.000 | 1.000 |
| operative* | | | | |
| Post- | 0.962 | 1.000 | 0.900 | 0.994 |
| operative | | | | |
| 6 years prior | 0.923 | 1.000 | 0.800 | 0.975 |
| 5 years prior | 0.962 | 1.000 | 0.900 | 0.988 |
| 4 years prior | 0.923 | 1.000 | 0.800 | 0.932 |
| 3 years prior | 0.923 | 1.000 | 0.800 | 0.969 |
| 2 years prior | 0.923 | 1.000 | 0.800 | 1.000 |
| 1 years prior | 0.923 | 1.000 | 0.800 | 0.981 |
| Diagnosis | 1.000 | 1.000 | 1.000 | 1.000 |

AUC = area under the ROC curve
*Pre-operative values were normalized to time-matched creatinine values; all other post-operative values were normalized to both creatinine and pre-operative values.
For all time-points except at 4 years prior to diagnosis the random forest classification provided the highest AUC.

TABLE 7
Predictive value of a full panel of all seven biomarkers at each
time-point investigated using Naïve Bayes, Logistic, Bagging,
and J48 Decision Tree classification strategies.

| Time- | | | | | |
|---|---|---|---|---|---|
| point | Classification | Accuracy | Sensitivity | Specificity | AUC |
| Pre- | Naive Bayes | 0.846 | 0.938 | 0.700 | 0.894 |
| operative | Logistic | 0.885 | 0.938 | 0.800 | 0.900 |
| | Bagging | 0.885 | 0.938 | 0.800 | 0.981 |
| | J48 Decision | 0.962 | 0.938 | 1.000 | 0.994 |
| | Tree | | | | |
| Post- | Naive Bayes | 0.692 | 0.388 | 0.700 | 0.750 |
| operative | Logistic | 0.962 | 1.000 | 0.900 | 0.944 |
| | Bagging | 0.731 | 0.875 | 0.500 | 0.837 |
| | J48 Decision | 0.769 | 0.688 | 0.900 | 0.822 |
| | Tree | | | | |
| 6 years | Naive Bayes | 0.692 | 0.688 | 0.700 | 0.813 |
| prior | Logistic | 0.846 | 0.938 | 0.700 | 0.900 |
| | Bagging | 0.808 | 0.875 | 0.700 | 0.866 |
| | J48 Decision | 0.808 | 1.000 | 0.500 | 0.788 |
| | Tree | | | | |
| 5 years | Naive Bayes | 0.692 | 0.625 | 0.800 | 0.806 |
| prior | Logistic | 0.731 | 0.813 | 0.600 | 0.831 |
| | Bagging | 0.808 | 0.938 | 0.600 | 0.847 |
| | J48 Decision | 0.769 | 0.750 | 0.800 | 0.788 |
| | Tree | | | | |
| 4 years | Naive Bayes | 0.731 | 0.813 | 0.600 | 0.863 |
| prior | Logistic | 0.808 | 0.813 | 0.800 | 0.875 |
| | Bagging | 0.808 | 1.000 | 0.500 | 0.950 |
| | J48 Decision | 0.885 | 0.938 | 0.800 | 0.959 |
| | Tree | | | | |
| 3 years | Naive Bayes | 0.654 | 0.563 | 0.800 | 0.738 |
| prior | Logistic | 0.923 | 1.000 | 0.800 | 0.969 |
| | Bagging | 0.731 | 0.875 | 0.500 | 0.803 |
| | J48 Decision | 0.615 | 1.000 | 0.000 | 0.500 |
| | Tree | | | | |
| 2 years | Naive Bayes | 0.731 | 0.750 | 0.700 | 0.900 |
| prior | Logistic | 0.692 | 0.750 | 0.600 | 0.813 |
| | Bagging | 0.808 | 0.938 | 0.600 | 0.919 |
| | J48 Decision | 0.846 | 0.875 | 0.800 | 0.925 |
| | Tree | | | | |
| 1 years | Naive Bayes | 0.615 | 0.625 | 0.600 | 0.744 |
| prior | Logistic | 0.808 | 0.900 | 0.600 | 0.813 |
| | Bagging | 0.808 | 0.938 | 0.600 | 0.881 |
| | J48 Decision | 0.885 | 1.000 | 0.700 | 0.928 |
| | Tree | | | | |
| Diagnosis | Naive Bayes | 0.846 | 0.750 | 1.000 | 0.975 |
| | Logistic | 0.923 | 0.875 | 1.000 | 0.950 |
| | Bagging | 0.923 | 0.875 | 1.000 | 0.991 |
| | J48 Decision | 0.962 | 0.938 | 1.000 | 0.969 |
| | Tree | | | | |

AUC = area under the receiver operating characteristic (ROC) curve

At diagnosis, a panel of α-CTX and IL-6 performed similarly to the panel including all seven biomarkers. Therefore, the two-marker panel was assessed at each time-point prior to diagnosis (Table 8). This limited panel performed comparably to the full panel with nearly the same accuracy sensitivity, specificity, and AUC.

TABLE 8
Accuracy, sensitivity, specificity, and AUC of a limited panel
comprised of α-CTX and IL-6 at each time-point investigated
using the Random Forest Classification method.

| Time-point | Accuracy | Sensitivity | Specificity | AUC |
|---|---|---|---|---|
| Pre- | 1.000 | 1.000 | 1.000 | 1.000 |
| operative* | | | | |
| Post- | 0.962 | 1.000 | 0.900 | 0.941 |
| operative | | | | |
| 6 years prior | 0.923 | 1.000 | 0.800 | 0.975 |
| 5 years prior | 0.962 | 1.000 | 0.900 | 0.988 |
| 4 years prior | 0.923 | 1.000 | 0.800 | 0.988 |
| 3 years prior | 0.923 | 1.000 | 0.800 | 0.969 |
| 2 years prior | 0.923 | 1.000 | 0.800 | 0.988 |
| 1 years prior | 0.923 | 1.000 | 0.800 | 0.981 |
| Diagnosis | 1.000 | 1.000 | 1.000 | 1.000 |

PPV = positive predictive value,
NPV = negative predictive value,
AUC = area under the ROC curve
*Pre-operative values were normalized to time-matched creatinine values; all other post-operative values were normalized to both creatinine and pre-operative values.
For all time-points prior to diagnosis the random forest classification provided the highest AUC.

TABLE 9
Predictive value of a limited panel comprised of α-CTX and IL-6
at each time-point investigated using Naïve Bayes, Logistic,
Bagging, and J48 Decision Tree classification strategies.

| Time- | | | | | |
|---|---|---|---|---|---|
| point | Classification | Accuracy | Sensitivity | Specificity | AUC |
| Pre- | Naive Bayes | 0.846 | 0.938 | 0.700 | 0.850 |
| operative | Logistic | 0.846 | 0.938 | 0.700 | 0.825 |
| | Bagging | 0.885 | 0.938 | 0.800 | 0.959 |
| | J48 Decision | 0.846 | 0.938 | 0.700 | 0.819 |
| | Tree | | | | |
| Post- | Naive Bayes | 0.538 | 0.563 | 0.500 | 0.528 |
| operative | Logistic | 0.615 | 1.000 | 0.000 | 0.663 |
| | Bagging | 0.692 | 0.875 | 0.400 | 0.794 |
| | J48 Decision | 0.615 | 1.000 | 0.000 | 0.500 |
| | Tree | | | | |
| 6 years | Naive Bayes | 0.577 | 0.563 | 0.600 | 0.666 |
| prior | Logistic | 0.654 | 0.813 | 0.400 | 0.688 |
| | Bagging | 0.769 | 0.938 | 0.500 | 0.800 |
| | J48 Decision | 0.769 | 0.875 | 0.600 | 0.775 |
| | Tree | | | | |
| 5 years | Naive Bayes | 0.769 | 0.750 | 0.800 | 0.759 |
| prior | Logistic | 0.692 | 0.813 | 0.500 | 0.781 |
| | Bagging | 0.885 | 1.000 | 0.700 | 0.894 |
| | J48 Decision | 0.769 | 0.750 | 0.800 | 0.788 |
| | Tree | | | | |
| 4 years | Naive Bayes | 0.538 | 0.438 | 0.700 | 0.681 |
| prior | Logistic | 0.692 | 0.688 | 0.700 | 0.769 |
| | Bagging | 0.731 | 0.938 | 0.400 | 0.897 |
| | J48 Decision | 0.731 | 1.000 | 0.300 | 0.706 |
| | Tree | | | | |
| 3 years | Naive Bayes | 0.538 | 0.438 | 0.700 | 0.712 |
| prior | Logistic | 0.654 | 0.688 | 0.600 | 0.756 |
| | Bagging | 0.846 | 0.938 | 0.700 | 0.872 |
| | J48 Decision | 0.615 | 1.000 | 0.000 | 0.500 |
| | Tree | | | | |
| 2 years | Naive Bayes | 0.500 | 0.438 | 0.600 | 0.650 |
| prior | Logistic | 0.615 | 0.688 | 0.500 | 0.725 |
| | Bagging | 0.769 | 0.938 | 0.500 | 0.831 |
| | J48 Decision | 0.769 | 0.938 | 0.500 | 0.813 |
| | Tree | | | | |
| 1 years | Naive Bayes | 0.692 | 0.75 | 0.600 | 0.731 |
| prior | Logistic | 0.731 | 0.813 | 0.600 | 0.794 |
| | Bagging | 0.769 | 0.938 | 0.500 | 0.881 |
| | J48 Decision | 0.808 | 0.875 | 0.700 | 0.847 |
| | Tree | | | | |
| Diagnosis | Naive Bayes | 0.808 | 0.75 | 0.900 | 0.947 |
| | Logistic | 0.885 | 0.875 | 0.900 | 0.900 |
| | Bagging | 0.808 | 0.688 | 1.000 | 0.984 |
| | J48 Decision | 0.962 | 1.000 | 0.900 | 0.994 |
| | Tree | | | | |

AUC = area under the ROC curve

### Discussion

The goal of the current study was to discover biomarkers that differentiate patients who developed osteolysis from those who did not develop osteolysis before conventional radiographic diagnosis. The largest between-group differences were noted in the pre-operative samples, while the largest AUC was found using the pre-operative biomarker levels of a panel of α-CTX and IL-6. Further, if normalized to pre-operative biomarker values, the AUC was 0.941 or greater at all post-operative time points using the two biomarker panel. Results from this study demonstrate the importance of pre-operative biomarker levels in identifying patients at risk for peri-implant osteolysis.

Peri-implant osteolysis is currently diagnosed via radiographic detection of peri-implant lesions, which are noticeable only after substantial bone loss. Revision surgery to replace failed implants is associated with worse outcomes than primary surgery⁷⁻⁹ with loss of bone mass prior to revision being a contributing factor¹⁰. Identification of a biomarker or biomarker panel that could detect ongoing peri-implant osteolysis prior to substantial bone loss or even serve as a risk factor prior to surgery would allow for early intervention or motivate more detailed clinical monitoring. In the current study, we used a unique biorepository of urine samples collected prospectively in patients receiving primary total hip replacements. The repository was developed as part of a longitudinal study of metal release and transport from THRs of different fixation modalities and designs^{24; 25}.

The biomarkers tested in this study were primarily identified by a systematic review of the literature¹⁷ and informed by previous work from our group using a rat model²⁶, which we used to prioritize 10 candidate biomarkers. Of these 10 markers, TRAP 5b, cathepsin K, RANKL, and osteocalcin were not detected in urine using conventional ELISA assays. α-CTX was added as a candidate biomarker that was not originally identified in our systematic review, but has been shown to have considerable promise as a biomarker for cancer-related osteolysis^{27; 28}. Of the seven biomarkers studied, α-CTX and IL-6, show the most promise for identifying at-risk patients.

Surprisingly, the biomarkers with the highest accuracy in identifying at risk patients were the pre-operative levels of α-CTX and IL-6. This result suggests that particular patients may be at greater risk for peri-implant osteolysis following THA. Indeed, genetic risk factors for susceptibility to osteolysis have recently been identified^{29; 30}. Similar to the current study, IL-6 has been implicated, with a rare haplotype within IL-6 being positively associated with osteolysis³¹ and single nucleotide polymorphisms in the IL-6 promotor have been reported to be predictive for aseptic loosening³². The addition of α-CTX as a predictive marker is in line with various studies that have demonstrated that pre-surgical remodeling rates contribute to the success of THA^{12; 33; 34}. Because the pre-operative values provided the highest AUC in both the full panel and the limited panel outcomes and only pre-operative normalized data showed post-operative differences between groups, it is likely that the pre-operative values drove many of the post-operative results. Thus, pre-existing patient factors may determine susceptibility to the development of particulate-induced osteolysis.

The panel approach considerably increased the accuracy of the urinary biomarkers, likely due to the multi-factorial nature of peri-implant osteolysis³⁵. The pathophysiology of peri-implant osteolysis involves an inflammatory response to wear particles, which activates bone resorption³⁶. Therefore, it is not surprising to find increased accuracy when combining a marker of bone resorption, α-CTX, and one of inflammation, IL-6, compared to either marker alone. He et al.³⁷ similarly found that the combination of six biomarkers, including markers associated with both bone resorption and inflammation showed larger between-group differences at the time of diagnosis compared to any single biomarker alone. In the current study, we found that the combination of α-CTX and IL-6 provided diagnostic accuracy comparable to a panel of seven biomarkers.

C-terminal telopeptide of collagen type I, crosslaps, or CTX-I is a cross-linked fragment released from the alpha I collagen chain by osteoclastic resorption of the bone matrix³⁸. CTX exists in two isoforms, α or β. Newly deposited collagen generally exists in the α, or non-isomerized form, which is isomerized to the β isoform as the bone matrix ages³⁹. β-CTX is a commonly measured biomarker of osteoclastic bone resorption used to predict bone mass loss, response to treatment, and risk of osteoporotic fractures⁴⁰. A previous study evaluating the utility of urinary β-CTX as a biomarker of peri-implant osteolysis reported no difference between groups⁴¹, which is consistent with results in the current study. α-CTX is emerging as a biomarker for osteolysis caused by the skeletal invasion of metastatic cancers, such as breast cancer^{27; 28}. α-CTX is thought to reflect pathological skeletal remodeling wherein new bone is resorbed before it is able to isomerize into the β isoform^{42; 43}. It is possible that the inflammatory response to particles causes resorption of new bone matrix, ultimately leading to the development of peri-implant osteolytic lesions. Urinary α-CTX has been previously studied in the context of peri-implant osteolysis at end-stage of the disease, but the authors noted no between-group differences⁴⁴ . In our study, α-CTX concentrations were significantly elevated at the point of radiographic osteolysis diagnosis. The reasons for this discrepancy are unknown, but may reflect differences in the study design, which include the use of second-morning void urine vs. 24-hour urine, or the increased average age of the patients evaluated in the study by Lawrence et al⁴⁴ .

IL-6 is released by macrophages in response to implant particles^{3; 45-50} and various studies have demonstrated an increase in the number of IL-6 positive cells from interfacial tissues retrieved from patients with failed implants⁵¹⁻⁵³. IL-6 has also been reported to be elevated in the serum of patients with osteolysis compared to controls⁵⁴. Similar to α-CTX, the individual predictive value of IL-6 was moderate, with the between-group differences only becoming significant at 1 year prior to diagnosis. However, when α-CTX and IL-6 were combined in a panel, these two biomarkers demonstrated high accuracy for identifying patients at risk for osteolysis.

The current study is one of only two that has investigated biomarkers longitudinally in THR patients and the first to do so in samples collected before clinical diagnosis of osteolysis. The other longitudinal study found that biomarker levels, specifically osteocalcin and cross-linked carboxyterminal telopeptide of type I collagen (ICTP), were elevated before implant migration as assessed by radiosteriometric analysis (RSA)¹⁸. Early implant migration is considered a risk factor for late loosening⁵⁵ and, therefore, the authors concluded that early elevation in these biomarkers is predictive of future migration. In our study, patients were categorized based on routine radiographic analysis and therefore, biomarker levels in the current study relate directly to the clinical manifestation of osteolysis. Taken together, these two studies suggest it may be possible to identify patients at risk for subsequent implant loosening prior to conventional diagnostic strategies.

Clinically, it is envisioned that biomarker measurements, once validated, could serve as a compliment to routine radiographic surveillance. Further, if patients at risk for subsequent osteolysis development could be identified at an early stage, this at-risk group could be designated for more intensive monitoring, in an effort to identify complications prior to substantial bone loss. Finally, although there are currently no validated treatment options for peri-implant osteolysis apart from revision surgery, several preclinical animal models have identified pharmacological agents able to prevent or reverse osteolysis if treatment is initiated early¹¹⁻¹³. If biomarkers are validated, it is possible that they could motivate future pharmaceutical clinical trials by identifying patients either at risk for osteolysis or early in disease progression, for recruitment into such trials. In addition, validated biomarkers can be used as a method to monitor response to any pharmacological intervention as they are more sensitive than radiographic studies.

We evaluated the use of urinary biomarkers to identify patients who developed peri-implant osteolysis prior to radiographic diagnosis. Although single biomarkers showed moderate accuracy, the combination of α-CTX, a bone resorption marker, and IL-6, an inflammatory marker, lead to high accuracy in the differentiation of patients who eventually developed peri-implant osteolysis from those with no signs of osteolysis.

The practice of the present invention will employ, unless otherwise indicated, conventional methods for measuring the level of the biomarker within the skill of the art. The techniques may include, but are not limited to, molecular biology and immunology. Such techniques are explained fully in the literature. See, e.g., Sambrook, et al. Molecular Cloning: A Laboratory Manual (Current Edition, Cold Spring Harbor Laboratory Press, Cold Spring Harbor, N.Y.); Current Protocols in Molecular Biology (Eds. A Ausubel et al., NY: John Wiley & Sons, Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition).

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

### REFERENCES

1. Kurtz S, Ong K, Lau E, et al. 2007. Projections of primary and revision hip and knee arthroplasty in the United States from 2005 to 2030. Journal of Bone and Joint Surgery 89:780-785.

2. Bozic KJ, Kurtz SM, Lau E, et al. 2009. The epidemiology of revision total hip arthroplasty in the United States. J Bone Joint Surg Am 91:128-133.

3. Hallab NJ, Jacobs JJ. 2009. Biologic effects of implant debris. BullNYUHospJtDis 67:182-188.

4. Purdue PE, Koulouvaris P, Potter HG, et al. 2007. The cellular and molecular biology of periprosthetic osteolysis. ClinOrthopRelat Res 454:251-261.

5. Cobelli N, Scharf B, Crisi GM, et al. 2011. Mediators of the inflammatory response to joint replacement devices. NatRevRheumatol 7:600-608.

6. Berkowitz JL, Potter HG. 2017. Advanced MRI Techniques for the Hip Joint: Focus on the Postoperative Hip. AJR Am J Roentgenol 209:534-543.

7. Mahomed NN, Barrett JA, Katz JN, et al. 2003. Rates and outcomes of primary and revision total hip replacement in the United States medicare population. JBone Joint SurgAm 85-A:27-32.

8. Katz JN, Losina E, Barrett J, et al. 2001. Association between hospital and surgeon procedure volume and outcomes of total hip replacement in the United States medicare population. JBone Joint SurgAm 83-A:1622-1629.

9. Katz JN, Phillips CB, Baron JA, et al. 2003. Association of hospital and surgeon volume of total hip replacement with functional status and satisfaction three years following surgery. Arthritis Rheum 48:560-568.

10. Panegrossi G, Ceretti M, Papalia M, et al. 2014. Bone loss management in total knee revision surgery. International Orthopaedics 38:419-427.

11. Ren K, Dusad A, Yuan F, et al. 2014. Macromolecular prodrug of dexamethasone prevents particle-induced peri-implant osteolysis with reduced systemic side effects. J Control Release 175:1-9.

12. Ross RD, Hamilton JL, Wilson BM, et al. 2014. Pharmacologic augmentation of implant fixation in osteopenic bone. CurrOsteoporosRep 12:55-64.

13. Moran MM, Wilson BM, Ross RD, et al. 2017. Arthrotomy-based preclinical models of particle-induced osteolysis: A systematic review. Journal of orthopaedic research: official publication of the Orthopaedic Research Society 35:2595-2605.

14. Wilkinson JM, Little DG. 2011. Bisphosphonates in orthopedic applications. Bone 49:95-102.

15. Thillemann TM, Pedersen AB, Mehnert F, et al. 2010. Postoperative use of bisphosphonates and risk of revision after primary total hip arthroplasty: a nationwide population-based study. Bone 46:946-951.

16. Garnero P. 2008. Biomarkers for osteoporosis management: utility in diagnosis, fracture risk prediction and therapy monitoring. MolDiagnTher 12:157-170.

17. Sumner DR, Ross R, Purdue E. 2014. Are there biological markers for wear or corrosion? A systematic review. Clinical Orthopaedics and Related Research 472:3728-3739.

18. Li MGmcuea, Thorsen K, Nilsson KG. 2004. Increased bone turnover as reflected by biochemical markers in patients with potentially unstable fixation of the tibial component. Archives of orthopaedic and trauma surgery 124:404-409.

19. DeLee JG, Charnley J. 1976. Radiological demarcation of cemented sockets in total hip replacement. Clin Orthop Relat Res:20-32.

20. Dorr LD, Bloebaum R, Emmanual J, et al. 1990. Histologic, biochemical, and ion analysis of tissue and fluids retrieved during total hip arthroplasty. Clin Orthop Relat Res:82-95.

21. Gruen TA, McNeice GM, Amstutz HC. 1979. Modes of failure of cemented stem-type femoral components: a radiographic analysis of loosening. Clinical Orthopaedics & Related Research 141:17-27.

22. Engh CA, Jr., Hopper RH, Jr., Huynh C, et al. 2012. A prospective, randomized study of cross-linked and non-cross-linked polyethylene for total hip arthroplasty at 10-year follow-up. The Journal of arthroplasty 27:2-7.e1.

23. Engh CA, Jr., Stepniewski AS, Ginn SD, et al. 2006. A randomized prospective evaluation of outcomes after total hip arthroplasty using cross-linked marathon and non-cross-linked Enduron polyethylene liners. The Journal of arthroplasty 21:17-25.

24. Levine BR, Hsu AR, Skipor AK, et al. 2013. Ten-year outcome of serum metal ion levels after primary total hip arthroplasty: a concise follow-up of a previous report*. J Bone Joint Surg Am 95:512-518.

25. Jacobs JJ, Skipor AK, Patterson LM, et al. 1998. Metal release in patients who have had a primary total hip arthroplasty. A prospective, controlled, longitudinal study. J Bone Joint Surg Am 80:1447-1458.

26. Ross RD, Virdi AS, Liu S, et al. 2014. Particle-induced osteolysis is not accompanied by systemic remodeling but is reflected by systemic bone biomarkers. Journal of Orthopaedic Research 32:967-973.

27. Cloos PA, Christgau S, Lyubimova N, et al. 2003. Breast cancer patients with bone metastases are characterised by increased levels of nonisomerised type I collagen fragments. Breast Cancer Res 5:R103-109.

28. Leeming DJ, Delling G, Koizumi M, et al. 2006. Alpha CTX as a biomarker of skeletal invasion of breast cancer: immunolocalization and the load dependency of urinary excretion. Cancer Epidemiol Biomarkers Prey 15:1392-1395.

29. Del Buono A, Denaro V, Maffulli N. 2012. Genetic susceptibility to aseptic loosening following total hip arthroplasty: a systematic review. BrMedBull 101:39-55.

30. Noordin S, Masri B. 2012. Periprosthetic osteolysis: genetics, mechanisms and potential therapeutic interventions. Canadian journal of surgery Journal canadien de chirurgie 55:408-417.

31. Gordon A, Kiss-Toth E, Stockley I, et al. 2008. Polymorphisms in the interleukin-1 receptor antagonist and interleukin-6 genes affect risk of osteolysis in patients with total hip arthroplasty. Arthritis Rheum 58:3157-3165.

32. Kolundzic R, Orlic D, Trkulja V, et al. 2006. Single nucleotide polymorphisms in the interleukin-6 gene promoter, tumor necrosis factor-alpha gene promoter, and transforming growth factor-betal gene signal sequence as predictors of time to onset of aseptic loosening after total hip arthroplasty: preliminary study. J Orthop Sci 11:592-600.

33. Alm JJ, Makinen TJ, Lankinen P, et al. 2009. Female patients with low systemic BMD are prone to bone loss in Gruen zone 7 after cementless total hip arthroplasty. Acta Orthop 80:531-537.

34. Aro HT, Alm JJ, Moritz N, et al. 2012. Low BMD affects initial stability and delays stem osseointegration in cementless total hip arthroplasty in women: a 2-year RSA study of 39 patients. Acta orthopaedica 83:107-114.

35. Beck RT, Illingworth KD, Saleh KJ. 2012. Review of periprosthetic osteolysis in total joint arthroplasty: an emphasis on host factors and future directions. JOrthopRes 30:541-546.

36. Abu-Amer Y, Darwech I, Clohisy JC. 2007. Aseptic loosening of total joint replacements: mechanisms underlying osteolysis and potential therapies. Arthritis ResTher 9 Suppl 1:S6.

37. He T, Wu W, Huang Y, et al. 2013. Multiple biomarkers analysis for the early detection of prosthetic aseptic loosening of hip arthroplasty. International Orthopaedics 37:1025-1031.

38. Cloos PA, Lyubimova N, Solberg H, et al. 2004. An immunoassay for measuring fragments of newly synthesized collagen type I produced during metastatic invasion of bone. Clin Lab 50:279-289.

39. Henriksen K, Leeming DJ, Byrjalsen I, et al. 2007. Osteoclasts prefer aged bone. Osteoporos Int 18:751-759.

40. Eastell R, Hannon RA. 2008. Biomarkers of bone health and osteoporosis risk. ProcNutrSoc 67:157-162.

41. Streich NA, Gotterbarm T, Jung M, et al. 2009. Biochemical markers of bone turnover in aseptic loosening in hip arthroplasty. International Orthopaedics 33:77-82.

42. Cloos PA, Fledelius C. 2000. Collagen fragments in urine derived from bone resorption are highly racemized and isomerized: a biological clock of protein aging with clinical potential. Biochem J 345 Pt 3:473-480.

43. Fledelius C, Johnsen AH, Cloos PA, et al. 1997. Characterization of urinary degradation products derived from type I collagen. Identification of a beta-isomerized Asp-Gly sequence within the C-terminal telopeptide (alphal) region. J Biol Chem 272:9755-9763.

44. Lawrence NR, Jayasuriya RL, Gossiel F, et al. 2015. Diagnostic accuracy of bone turnover markers as a screening tool for aseptic loosening after total hip arthroplasty. Hip international: the journal of clinical and experimental research on hip pathology and therapy 25:525-530.

45. Purdue PE, Koulouvaris P, Nestor BJ, et al. 2006. The central role of wear debris in periprosthetic osteolysis. HSS journal: the musculoskeletal journal of Hospital for Special Surgery 2:102-113.

46. Blaine TA, Rosier RN, Puzas JE, et al. 1996. Increased levels of tumor necrosis factor-alpha and interleukin-6 protein and messenger RNA in human peripheral blood monocytes due to titanium particles. J Bone Joint Surg Am 78:1181-1192.

47. Nakashima Y, Sun DH, Trindade MC, et al. 1999. Signaling pathways for tumor necrosis factor-alpha and interleukin-6 expression in human macrophages exposed to titanium-alloy particulate debris in vitro. J Bone Joint Surg Am 81:603-615.

48. Wimhurst JA, Brooks RA, Rushton N. 2001. Inflammatory responses of human primary macrophages to particulate bone cements in vitro. J Bone Joint Surg Br 83:278-282.

49. Ingham E, Green TR, Stone MH, et al. 2000. Production of TNF-alpha and bone resorbing activity by macrophages in response to different types of bone cement particles. Biomaterials 21:1005-1013.

50. Maloney WJ, James RE, Smith RL. 1996. Human macrophage response to retrieved titanium alloy particles in vitro. Clin Orthop Relat Res:268-278.

51. Chiba J, Rubash HE, Kim KJ, et al. 1994. The characterization of cytokines in the interface tissue obtained from failed cementless total hip arthroplasty with and without femoral osteolysis. Clin Orthop Relat Res:304-312.

52. Goodman SB, Huie P, Song Y, et al. 1998. Cellular profile and cytokine production at prosthetic interfaces. Study of tissues retrieved from revised hip and knee replacements. JBone Joint SurgBr 80:531-539.

53. Stea S, Visentin M, Granchi D, et al. 2000. Cytokines and osteolysis around total hip prostheses. Cytokine 12:1575-1579.

54. Hernigou P, Intrator L, Bahrami T, et al. 1999. Interleukin-6 in the blood of patients with total hip arthroplasty without loosening. ClinOrthopRelat Res:147-154.

55. Ryd L. 1986. Micromotion in knee arthroplasty. Acta Orthopaedica Scandinavica 57 (suppl).

56. Lester G. 2008. Clinical research in OA—the NIH Osteoarthritis Initiative. J Musculoskelet Neuronal Interact 8:313-314.

57. Peterfy CG, Schneider E, Nevitt M. 2008. The osteoarthritis initiative: report on the design rationale for the magnetic resonance imaging protocol for the knee. Osteoarthritis Cartilage 16:1433-1441.

58. Hunter DJ, Nevitt M, Losina E, et al. 2014. Biomarkers for osteoarthritis: current position and steps towards further validation. Best Pract Res Clin Rheumatol 28:61-71.

1. A method for measuring a panel of biomarkers in a subject suspected of being at risk for peri-implant osteolysis, the method comprising:
obtaining a biological sample from the subject; and
measuring a level of at least two biomarkers in a biomarker panel in the sample, wherein the biomarker panel comprises α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX).

2. The method according to claim 1, wherein the at least two biomarkers are α-CTX and IL-6.

3. The method according to claim 1, wherein the at least two biomarkers are measured prior to an implant surgery.

4. The method according to claim 1, wherein the at least two biomarkers are normalized to creatinine levels of the subject.

5. The method according to claim 1, wherein the at least two biomarkers are measured post-implant surgery and are normalized to pre-implant levels of the at least two biomarkers.

6. The method according to claim 1, wherein the biological sample comprises a urine sample.

7. The method according to claim 1, comprising measuring the level of each biomarker in the biomarker panel selected from the group consisting of α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX).

8. The method according to claim 7, wherein each biomarker is measured prior to an implant surgery.

9. The method according to claim 7, wherein each biomarker is normalized to creatinine levels of the subject.

10. The method according to claim 7, wherein each biomarker is measured post-implant surgery and each biomarker is normalized to its pre-implant level.

11. A kit for performing measurement of the levels of the at least two biomarkers of the subject in claim 1, wherein the kit comprises reagents for measuring the at least two biomarkers.

12. The kit according to claim 11, wherein the kit comprises reagents for ELISA measurements of the at least two biomarkers.

13. The kit according to claim 11, wherein the kit comprises reagents for measuring α-CTX and IL-6.

14. The kit according to claim 13, wherein the kit further comprises reagents for measuring β-CTX, IL-8, OPG, DPD, and NTX.

15. A method for treating a subject at risk for or having peri-implant osteolysis, the method comprising:
obtaining or having obtained a biological sample from the subject;
and
performing or having performed a measurement of a level of at least two biomarkers in a biomarker panel in the sample, wherein the biomarker panel comprises α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX); and
administering to the subject at risk for or having peri-implant osteolysis a therapeutically effective amount of a therapeutic agent selected from the group consisting of anti-catabolic agents to reduce further bone loss or anabolic agents to increase bone formation around the implant.

16. The method of claim 15, wherein the at least two biomarkers are α-CTX and IL-6.

17. The method according to claim 15, wherein the at least two biomarkers are measured prior to an implant surgery.

18. The method according to claim 15, wherein the at least two biomarkers are normalized to creatinine levels of the subject.

19. The method according to claim 15, wherein the at least two biomarkers are measured post-implant surgery and are normalized to pre-implant levels of the at least two biomarkers.

20. The method according to claim 15, comprising measuring the level of each biomarker in the biomarker panel selected from the group consisting of α-crosslaps (α-CTX), β-crosslaps (β-CTX), Interleukin-6 (IL-6), Interleukin-8 (IL-8), osteoprotegrin (OPG), deoxypyridinoline (DPD), and cross-linked N-telopeptides (NTX).
